(19) United States
(12) Patent Application Publication
(10) Pub. No.: US 2024/0024449 A1
(43) Pub. Date: Jan. 25, 2024

(54) METHODS AND COMPOSITIONS FOR IMPROVING IMMUNE SYSTEM FUNCTION

(71) Applicant: Immunology Diagnostics, LLC, Los Angeles, CA (US)

(72) Inventor: Bruce S. GILLIS, Beverly Hills, CA (US)

(21) Appl. No.: 18/354,838

(22) Filed: Jul. 19, 2023

Related U.S. Application Data
(60) Provisional application No. 63/390,766, filed on Jul. 20, 2022.

(51) Int. Cl. A61K 39/04; A61P 37/04

(52) U.S. Cl. CPC A61K 39/04; A61P 37/04; A61K 2039/521

(57) ABSTRACT

The invention provides methods and compositions for modulating the immune system of an individual. The methods provided herein entail administering a composition comprising a killed or inactivated Mycobacterium or antigenic fragments derived therefrom. Also provided herein are methods for assessing alteration of immune system functioning following administration of a composition comprising aa killed or inactivated Mycobacterium or antigenic fragments derived therefrom.

### CROSS REFERENCE TO RELATED APPLICATIONS

This application claims benefit from U.S. Provisional Application No. 63/390,766 filed Jul. 20, 2022, which is incorporated by reference herein in its entirety for all purposes.

### FIELD OF THE INVENTION

This invention provides methods for treating immune deficiency diseases (e.g. fibromyalgia) using compositions comprising isolated Mycobacteria or antigenic fragments derived therefrom.

### BACKGROUND OF THE INVENTION

Mycobacteria have diverse and established significant roles in modulating immune system responses. This includes the recognition of the impact of the Bacillus-Calmette-Guerin (BCG) vaccine which is derived from Mycobacterium bovis. A number of studies have also shown that the Mycobacterial cell wall can stimulate the immune system and it has also been documented in killing cancer cells.

Because of the documented potential side effects of BCG, there is a need in the art for the discovery of other species of Mycobacteria that are capable of modulating the functioning of the immune system, and subsequent use of said other Mycobacterial species in subjects suffering from diseases or disorders for which abnormal immune system functioning is causal.

### SUMMARY OF THE INVENTION

In one aspect, provided herein is a method of modulating immune system function in a subject, comprising administering a composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof. In some cases, the Mycobacterium is live-attenuated. In some cases, the Mycobacterium is heat-killed. In some cases, the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023. In some cases, the composition is formulated as a nutritional supplement and/or post-biotic. In some cases, the nutritional supplement and/or post-biotic is incorporated into a food or beverage. In some cases, the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some cases, the composition comprises from 2×10⁷ to 5×10⁹ colony forming units (CFUs) of the Mycobacterium. In some cases, the composition comprises at least about 5×10⁶ CFU of the Mycobacterium. In some cases, the composition comprises 1 mg, 10 mg, 100 mg or 250 mg of the Mycobacterium. In some cases, the composition is administered in repeat doses. In some cases, the modulation in immune system function is evidenced by production and/or secretion of TH1 cytokines, upregulation of granzyme B or both as compared to a control. In some cases, the modulation in immune system function is evidenced by production and/or secretion of cytokines selected from the group consisting of interluekin-6 (IL-6), interluekin-8 (IL8), macrophage inflammatory protein-1 alpha (MIP-1alpha), macrophage inflammatory protein-1 beta (MIP-1beta) and any combination thereof as compared to a control. In some cases, the control is a subject that has not been administered the composition. In some cases, the subject suffers from a disease or disorder caused by a deficiency or abnormality in immune system function. In some cases, the subject suffers from a disease or disorder selected from the group consisting of fibromyalgia, chronic fatigue syndrome, chronic pain, cancer interstitial cystitis, a primary immune deficiency disease (PIDD), depression, anxiety, diabetes, long COVID, brain fog, sleep disturbances or disorders, autoimmune diseases and inflammatory diseases.

In one aspect, provided herein is a composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof. In some cases, the Mycobacterium is live-attenuated. In some cases, the Mycobacterium is heat-killed. In some cases, the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023. In some cases, the composition is formulated as a nutritional supplement and/or post-biotic. In some cases, the nutritional supplement and/or post-biotic is incorporated into a food or beverage. In some cases, the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some cases, the composition comprises from 2×10⁷ to 5×10⁹ colony forming units (CFUs) of the Mycobacterium. In some cases, the composition comprises at least about 5×10⁶ CFU of the Mycobacterium. In some cases, the composition comprises 1 mg, 10 mg, 100 mg or 250 mg of the Mycobacterium.

### DETAILED DESCRIPTION OF THE INVENTION

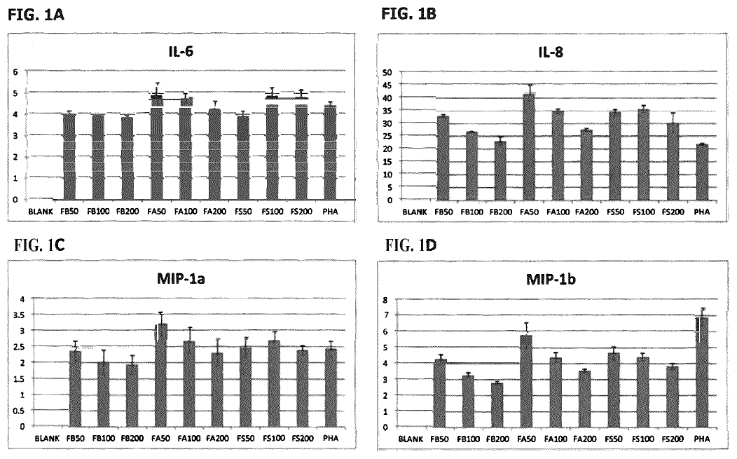

FIGS. 1A-1D illustrate extracellular cytokine and chemokine expression in PBMC cultures challenged to M. smegmatis isolates. Concentrations of IL-6 (FIG. 1A), IL-8 (FIG. 1B), MIP1-alpha (FIG. 1C) and MIP1-beta (FIG. 1D) were measured in PBMC tissue culture supernatants isolated from PBMCs challenged with either 50 μg/ml (designated 50 in FIGS. 1A-1D), 100 μg/ml (designated 100 in FIGS. 1A-1D) or 200 μg/ml (designated 200 in FIGS. 1A-1D) of M. smegmatis isolates. PBMC cultures were challenged with the following isolates of M. smegmatis from feline: FB, an abscess; FA, abdomen; FS, skin. Concentrations of proteins are in ng/ml. Blank=the control PBMC culture; PHA=phytohemagglutinin (positive control; 10 μg/ml).

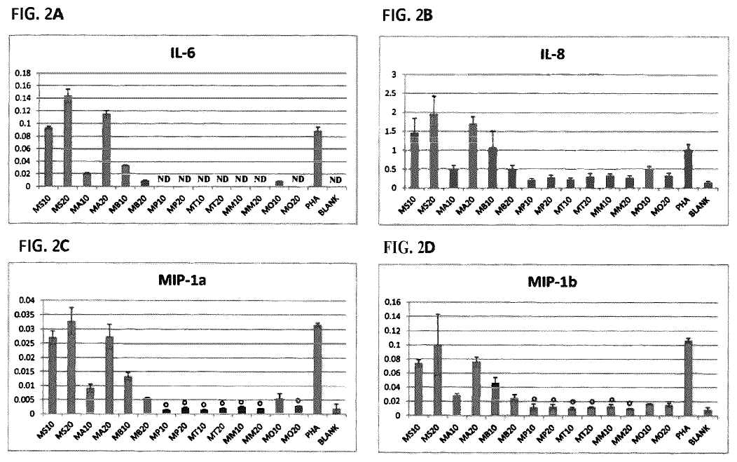

FIGS. 2A-2D illustrate extracellular cytokine and chemokine expression in PBMC cultures challenged to Mycobacterium strains. Concentrations of IL-6 (FIG. 2A), IL-8 (FIG. 2B), MIP1-alpha (FIG. 2C) and MIP1-beta (FIG. 2D) were measured in PBMC culture supernatants isolated from PBMCs challenged with either 50 μg/ml (designated 10 in FIGS. 2A-2D), 100 μg/ml (designated 20 in FIGS. 2A-2D) of Mycobacterial strains. The Mycobacterial strains were M. agri, ATCC27406 (MA); M. phlei, ATCC11758 (MP); M tokaiense, ATCC27282 (MT); M. smegmatis, ATCC19420 (MS); M. brumae, ATCC51384 (MB); M. aurum, ATCC23366 (MM); M. obuense, ATCC27023 (MO). Concentrations of proteins are in ng/ml. Blank=media only (negative control); PHA=phytohemagglutinin (positive control; 10 μg/ml). ND, not detected; o, denotes no significant differences from Blank.

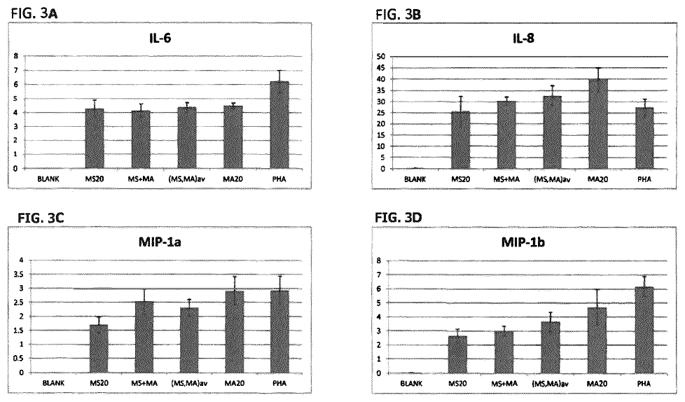

FIGS. 3A-3D illustrate PBMC responses to the mixtures of M. smegmatis with M. agri. Concentrations of IL-6 (FIG. 3A), IL-8 (FIG. 3B), MIP1-alpha (FIG. 3C) and MIP1-beta (FIG. 3D) measured in PBMC culture supernatants isolated from PBMCs challenged with 100 μg/ml (designated 20 in FIGS. 3A-3D) of M. smegmatis (MS) or M. agri (MA) or a combination of the two, each at 50 μg/ml (i.e., MS+MA in FIGS. 3A-3D). (MS, MA)av illustrates the cumulative average level for the respective cytokine in FIGS. 2A-2D for individual challenges with MS or MA. Concentrations of proteins are in ng/ml. Blank=media only (negative control); PHA=phytohemagglutinin (positive control; 10 μg/ml).

FIGS. 4A-4D illustrate PBMC responses to the mixtures of B. subtilis with either M smegmatis or M. agri. Concentrations of IL-6 (FIG. 4A), IL-8 (FIG. 4B), MIP-la (FIG. 4C), and MIP-10 (FIG. 4D) in PBMC cultures are shown. PBMC cultures were challenged to the following strains: BS100, 100 μg/ml B. subtilis; MS50+BS50, the mixture of 50 μg/ml M. smegmatis with 50 μg/ml B. subtilis; MA50+BS50, the mixture of 50 μg/ml M. agri with 50 g/ml B. subtilis. (MS100, BS100)av, the average of two protein concentrations in individual challenges to either 100 μg/ml M. smegmatis or 100 μg/ml B. subtilis. (MA100, BS100)av, the average of two protein concentrations in individual challenges to either 100 μg/ml M. agri or 100 μg/ml B. subtilis. The numbers above the bars denote p-values for increases in protein concentrations in combined challenges vs. the averages of two concentrations in individual challenges.

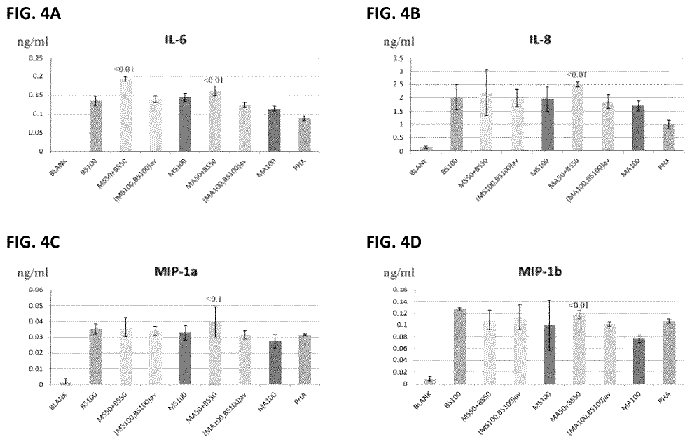

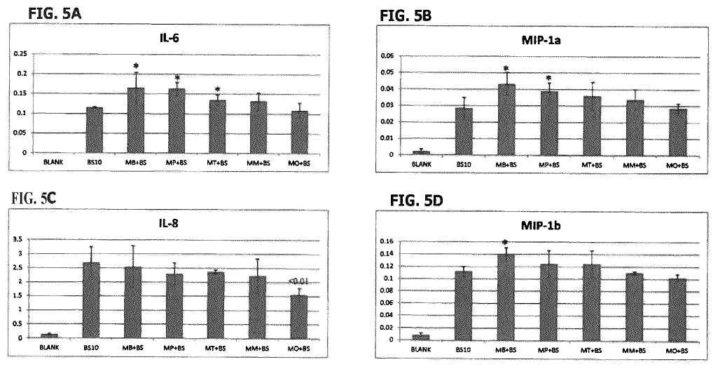

FIGS. 5A-5D illustrate PBMC responses to combinations of B. subtilis with various Mycobacterium strains. Concentrations of IL-6 (FIG. 5A), MIP1-alpha (FIG. 5B), IL-8 (FIG. 5C) and MIP1-beta (FIG. 5D) measured in PBMC culture supernatants isolated from PBMCs challenged with the mixtures of 50 μg/ml B. subtilis with the following strains: BS10, none; MB+BS, 50 μg/ml M. brumae; MP+BS, 50 μg/ml M. phlei; MT+BS, 50 μg/ml M. tokaiense; MM+BS, 50 μg/ml M. aurum; MO+BS, 50 μg/ml M. obuense. The number above the bar denotes the p-value for a decrease in IL-8 concentration in the combined MO+BS challenge vs. the B. subtilis challenge. Asterisks indicate statistically significant increases in protein concentrations in combined challenges vs. the B. subtifis challenge. Concentrations of proteins are in ng/ml. Blank=media only (negative control); PHA=phytohemagglutinin (positive control; 10 μg/ml).

### DETAILED DESCRIPTION OF THE INVENTION

### Definitions

Unless otherwise defined, all technical terms used herein have the same meaning as commonly understood by one of ordinary skill in the art to which this invention belongs.

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, “protein” and “polypeptide” are used synonymously to mean any peptide-linked chain of amino acids, regardless of length or post-translational modification, e.g., glycosylation or phosphorylation.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The term “cytokine” as used herein refers to small proteins that are secreted by specific cells of the immune system and glial cells, and include lymphokines, interleukins, and chemokines and their corresponding receptors, such as but not limited to IL-1, IL-2, IL-4, IL-5, IL-6, IL-7, IL-8, IL-10, IL-12, IL-13, IL-15, IL-17, IL-21, IFN-7, IFN-α, TNF-α, IP-10, MCP-1, MIG, MIP-la, MIP-10, GM-CSF, Eotaxin, RANTES, etc. In another aspect, the invention further includes determining the levels of one or more of IL-iRA, IL2R, IL-7, IL-12 (p40/p70), IL-13, IL-15, IL-17, IFN-α, IP-10, MIG, VEGF, G-CSF, EGF, FGF-basic and HGF. In yet another aspect, the invention also includes determining the levels of IL-9 and PDGF-BB or a combination thereof. The cytokine may be inflammatory or anti-inflammatory. In one embodiment, the cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of cytokine assayed maybe a mRNA, miRNA, or DNA.

As used herein, the term “treatment” is defined as the application or administration or use of a composition described herein, or identified by a method described herein, to a patient, or application or administration or use of the composition to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient”, “subject” and “individual” are used interchangeably herein, and can mean an animal. The animal can be any vertebrate or invertebrate. The animal can be any mammalian or non-mammalian animal. In some cases, the animal is selected from the group consisting of humans, canines, rodents, porcine, equines and felines. In one embodiment, the animal is mammalian, with human patients being preferred. In some cases, the animal is selected from the group consisting of birds, reptiles, insects, worms and fish. In some cases, the animal is amphibian. The animal can be selected from the group consisting of toads, frogs and salamanders. In some cases, the animal is a marsupial. The animal can be selected from the group consisting of wombats, wallabies, bandicoots, kangaroos, opossums and koalas. In some cases, the methods of the invention find use in experimental animals, in veterinary applications, and in the development of animal models for disease, including, but not limited to, rodents including mice, rats, and hamsters, as well as primates. In some cases, the methods of the invention find use in veterinary applications, wherein the subject is an animal as provided herein.

By the phrases “therapeutically effective amount” and “effective dosage” and “effective amount” is meant an amount sufficient to produce a desirable result; the exact nature of the result will vary depending on the nature of the disorder for which an individual is afflicted with or diagnosed to possess. For example, administration can the result in an increase in the expression or production of a cytokine or chemokine in an individual as compared to cytokine or chemokine production in the individual prior to administration and/or a control individual who either has not been administered or used the composition and is generally healthy or suffers from a similar or the same malady as the individual administered or using the composition. The skilled artisan will appreciate that certain factors can influence the dosage and timing required to effectively treat a subject, including but not limited to the severity of the disease or disorder, previous treatments, the general health and/or age of the subject, and other diseases present. Moreover, administration of a composition provided herein by a subject can include a single administration or a series of administrations.

The term “post-biotic” as herein can refer to microorganisms that have been killed by heat or inactivated by other means and are delivered to an individual as inactivated, non-replicating and/or dead cells. The ideal post-biotic is one that retains all or substantially all of the properties of the live microorganism but has completely and irrevocably lost the ability to replicate itself.

### Overview

The present invention provides compositions and methods for modulating the immune system of an individual. The modulation can be increasing the functioning of the immune system of the individual. The modulation can be decreasing the function of the immune system of the individual. The modulation can be increasing the expression of some cytokines or chemokines, while also decreasing the expression of other cytokines or chemokines. In one embodiment, provided herein is a composition comprising one or more Mycobacterial cells, wherein administration of said composition to an individual serves to modulate the functioning of the immune system of said individual. In another embodiment, provided herein is a method for improving the functioning of an individual's immune system by administering a composition provided herein, wherein the composition comprises one or more Mycobacterial cells. The one or more Mycobacterial cells present in a composition provided herein can be live-attenuated or heat-killed. In another embodiment, the one or more Mycobacterial cells present in a composition provided herein is a non-pathogenic Mycobacterial species. The non-pathogenic Mycobacterium can be live-attenuated or heat-killed. The individual may have been previously diagnosed with or may be suspected of suffering from or being afflicted with a malady (which can be a disease or disorder) known in the art and/or provided herein for which a deficiency or abnormality in immune system functioning is characterized, causal and/or plays a role.

Examples of maladies (also called conditions, diseases or disorders) that an individual may suffer from or be suspected of suffering from can be selected from the group consisting of fibromyalgia, chronic fatigue syndrome, chronic pain, ADHD, cancer, interstitial cystitis, a primary immune deficiency disease (PIDD), brain fog, complications from SARS-CoV2 (COVID) infection (e.g., long COVID, ARDS), sleep disturbances (e.g., sleep apnea), depression, anxiety, diabetes, autoimmune diseases and inflammatory disorders.

Severe acute respiratory syndrome coronavirus (SARS-CoV) is an enveloped virus with a single stranded positive-sense RNA genome (30 kb) that causes dangerous and potentially fatal conditions such as acute respiratory distress syndrome (ARDS) and acute lung injury (ALI) and long COVID. ARDS/ALI is a major public health problem, and in spite of significant advances in understanding of the pathophysiology of the disease, mortality and morbidity remain high. Long COVID may be broadly defined as signs, symptoms, and conditions that continue or develop after initial COVID-19 or SARS-CoV-2 infection. The signs, symptoms, and conditions may be present four weeks or more after the initial phase of infection; may be multisystemic; and may present with a relapsing—remitting pattern and progression or worsening over time, with the possibility of severe and life-threatening events even months or years after infection. Long COVID may not be considered as one condition. It may represent many potentially overlapping entities, likely with different biological causes and different sets of risk factors and outcomes. Long COVID may be referred to herein as many names, including Post-COVID Conditions, long-haul COVID, post-acute COVID-19, long-term effects of COVID, and chronic COVID. The term post-acute sequelae of SARS CoV-2 infection (PASC) may also used to refer to a subset of Long COVID. The fatality rate of SARS-CoV from November 2002 to July 2003 was 9.6% according to the World Health Organization. The fatality rate for CoV-2 is yet to be accurately determined, but it is anticipated that coronaviruses may occur cyclically, like the flu. A recent study that systematically mapped the interaction landscape between SARS-CoV-2 proteins and human proteins revealed that SARS-CoV-2 interacts with multiple innate immune pathways, including members of the inflammasome pathway (Gordon et al., 2020).

Long COVID can have a wide range of symptoms that can last weeks, months, or even years after infection. Sometimes the symptoms can even go away and come back again. For some people, Long COVID can last weeks, months, or years after COVID-19 illness and can sometimes result in disability. Subjects with Long COVID may experience health problems from different types and combinations of symptoms happening over different lengths of time.

General symptoms associated with long COVID can include: tiredness or fatigue that interferes with daily life, symptoms that get worse after physical or mental effort (also known as “post-exertional malaise”), fever, respiratory and heart symptoms, difficulty breathing or shortness of breath, cough, chest pain, fast-beating or pounding heart (also known as heart palpitations), neurological symptoms, difficulty thinking or concentrating (sometimes referred to as “brain fog”), headache, sleep problems, dizziness when you stand up (lightheadedness), pins-and-needles feelings, change in smell or taste, depression or anxiety, digestive symptoms, diarrhea, stomach pain, joint or muscle pain, rash, and/or changes in menstrual cycles. In some case, the symptoms associated with long COVID may be similar to those reported by people with myalgic encephalomyelitis/chronic fatigue syndrome (ME/CFS) and other poorly understood chronic illnesses that may occur after other infections.

In one embodiment, the compositions and methods provided herein may be administered to and/or be useful for modulating the immune system of an individual suffering from Long ARDS, ALI and/or long COVID.

Non-limiting examples of cancers that an individual who is administered a composition described herein may be suffering from or suspected of suffering include, but are not limited to: cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is a melanoma (e.g., metastatic malignant melanoma), renal cancer (e.g. clear cell carcinoma), prostate cancer (e.g. hormone refractory prostate adenocarcinoma), pancreatic cancer (e.g., adenocarcinoma), breast cancer, colon cancer, gallbladder cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, the cancer is a solid tumor cancer.

Non-limiting examples of autoimmune diseases and/or inflammatory diseases that an individual who is administered a composition described herein may be suffering from or suspected of suffering include, but are not limited to: arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis; inflammatory hyperproliferative skin diseases; psoriasis, such as plaque psoriasis, gutatte psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic faciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; chorioiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodosum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired spenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic re-perfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis.

Alternatively, non-limiting examples of autoimmune diseases and/or inflammatory diseases that an individual who is administered a composition described herein may be suffering from or suspected of suffering include, but are not limited to: Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Anti-TBM nephritis, Antiphospholipid syndrome, arthofibrosis, atrial fibrosis, autoimmune angioedema, autoimmune aplastic anemia, autoimmune dusautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenic purpura, autoimmune thyroid disease, autoimmune urticaria, axonal and neuronal neuropathies, Balo disease, Behcet's disease, benign mucosal pemphigold, Bullous pemphigold, cardiomyopathy, Castleman disease, Celiac Disease, Chagas disease, chronic fatigue syndrome, chronic inflammatory demyelinating polyneuropathy, chronic Lyme disease, chronic recurrent multifocal osteomyelitis, Churg-Strauss syndrome, cicatricial pemphigold, cirrhosis, Cogans syndrome, cold agglutinin disease, congenital heart block, Coxsackle myocarditis, CREST disease, Crohn's disease, Cystic Fibrosis, essential mixed cryoglobulinemia, deficiency of the interleukin-1 receptor antagonist, demyelinating neuropathies, dermatitis herpetiformis, dermatomyosis, Devic's disease, discoid lupus, Dressler's syndrome, Dupuytren's contracture, endometriosis, endomyocardial fibrosis, eosinophilic esophagitis, eosinophilic facsciitis, erythema nodosum, experimental allergic encephalomyelitis, Evans syndrome, Familial Mediterranean Fever, fibromyalgia, fibrosing alveolitis, giant cell arteritis, giant cell myocarditis, glomerulonephritis, Goodpasture's syndrome, Graft-versus-host disease (GVHD), granulomatosus with polyanglitis, Graves' disease, Guillain-Bare syndrome, Hashimoto's encephalitis, Hashimoto's thyroiditis, hemolytic anemia, Henoch-Schonlein purpura, hepatitis, herpes gestationis, hypogammaglobulinemia, idiopathic thrombocytopenic purpura, IgA nephropathy, IgG4-related sclerosing disease, immunoregulatory lipoproteins, inclusion body myositis, inflammatory bowel disorders, interstitial cystitis, juvenile arthritis, juvenile myositis, Kawasaki syndrome, keloid, Lambert-Eaton syndrome, leukocytoclastic vasculitis, lichen planus, lichen sclerosus, ligneous conjunctivitis, linear IgA disease, mediastinal fibrosis, Meniere's disease, microscopic polyanglitis, mixed connective tissue disease, Mooren's ulcer, Mucha-Hamermann disease, Multiple Sclerosis (MS), Myasthenia gravis, myelofibrosis, Myositis, narcolepsy, Neonatal Onset Multisystem Inflammatory Disease, nephrogenic systemic fibrosis, neutropenia, nonalcoholic fatty liver disease, nonalcoholic steatohepatitis (NASH), ocular-cicatricial pemphigold, optic neuritis, palindromic rheumatism, Pediatric Autoimmune Neuropsychiatric Disorders Associated with Streptococcus (PANDAS), paraneoplastic cerebellar degeneration, paroxysmal nocturnal nemoglobinuria, Parry Romberg syndrome, Parsonnage-Turner syndrome, Pars planitis, Pemphigus, Peripheral neuropathy, perivenous encephalomyelitis, pernicious anemia, Peyronie's disease, POEMS syndrome, polyarteritis nodosa, progressive massive fibrosis, Tumor Necrosis Factor Receptor-assoicated Periodic Syndrome, Type I autoimmune polyglandular syndrome, Type II autoimmune polyglandular syndrome, Type III autoimmune polyglandular syndrome, polymyalgia rhematica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arthritis, temporal arteritis, thrombocytopenic purpura, Tolosa-Hunt syndrome, transverse myelitis, Type 1 diabetes, ulcerative colitis, undifferentiated connective tissue disease, uveitis, vasculitis, vesiculobullous dermatosis, and Vitiligo.

Primary Immunodeficiency Diseases (PIDD) are a group of more than 150 diseases in which part of a subject's immune system is missing or does not function normally. To be considered a primary immunodeficiency, the cause of the immune deficiency must not be secondary in nature (e.g., caused by other disease, drug treatment, or environmental exposure to toxins). Most primary immunodeficiencies are genetic disorders and are diagnosed in children, although less severe forms may not be recognized until adulthood. About 1 in 500 people are born with a primary immunodeficiency. Non-limiting examples of primary immune deficiency diseases (PIDDs) that an individual who is administered a composition described herein may be suffering from or suspected of suffering include, but are not limited to: Autoimmune lymphoproliferative syndrome (ALPS), Autoimmune polyglandular syndrome type 1 (APS-1), BENTA disease, Caspase Eight Deficiency State (CEDS), CARD9 deficiency, Chronic Granulomatous Disease (CGD), Wiskott-Aldrich Syndrome (WAS), Severe Combined Immunodeficiency (SCID), PI3 Kinase Disease, LRBA Deficiency, DOCK8 deficiency and GATA2 Deficiency.

In one embodiment, administration, as defined herein, includes the administration of a composition provided herein in multiple aliquots and/or doses and/or on separate occasions. For example, administration can be one or times a day, semi-weekly, weekly, bi-monthly, monthly, semi-annually or annually.

In one aspect of the present invention, the Mycobacterium comprises a live-attenuated strain of a Mycobacterial species. In another aspect of the present invention the Mycobacterium comprises heat-killed strain of a Mycobacterial species.

Mycobacterial species for use in the compositions and/or methods of the present invention include, but are not limited to M. agri, M. brumae, M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. indicus pranii, M. tuberculosis, M. microti; M. africanum; M. kansasii, M. marinum; M simiae; M. gastri; M. nonchromogenicum; M. terrae; M. triviale; M. gordonae; M scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium; M. bovis and combinations thereof.

In one embodiment, the Mycobacterium is non-pathogenic. The non-pathogenic Mycobacterium can be heat-killed. The non-pathogenic Mycobacterium can be selected from M. agri, M. phlei, M. tokaiense, M. smegmatis, M. brumae, M. aurum, M. obuense and combinations thereof. In one embodiment, the non-pathogenic Mycobacterium is a rough variant. In one embodiment, the non-pathogenic Mycobacterium is a smooth variant. In one embodiment, the methods provided herein comprise administering a composition comprising M. obuense. Further to this embodiment, the M. obuense can be heat-inactivated. Further still to this embodiment, the M. obuense can be strain NCTC13365. Even further still to this embodiment, the composition comprising M. obuense is IMM-101. IMM-101 is a suspension of heat-killed whole cell Mycobacterium obuense. In one embodiment, the M. obuense can be strain ATCC27023. In one embodiment, the methods provided herein comprise administering a composition comprising M. agri. Further to this embodiment, the M. agri can be heat-inactivated. Further still to this embodiment, the M. agri can be strain ATCC27406. In one embodiment, the methods provided herein comprise administering a composition comprising M. phlei. Further to this embodiment, the M. phlei can be heat-inactivated. Further still to this embodiment, the M. phlei can be strain ATCC11758. In one embodiment, the methods provided herein comprise administering a composition comprising M. smegmatis. Further to this embodiment, the M. smegmatis can be heat-inactivated. Further still to this embodiment, the M. smegmatis can be strain ATCC19420. In one embodiment, the methods provided herein comprise administering a composition comprising M. tokaiense. Further to this embodiment, the M. tokaiense can be heat-inactivated. Further still to this embodiment, the M. tokaiense can be strain ATCC27282. In one embodiment, the methods provided herein comprise administering a composition comprising M. brumae. Further to this embodiment, the M. brumae can be heat-inactivated. Further still to this embodiment, the M. brumae can be strain ATCC51384. In one embodiment, the methods provided herein comprise administering a composition comprising M. aurum. Further to this embodiment, the M. aurum can be heat-inactivated. Further still to this embodiment, the M. aurum can be strain ATCC23366.

In one embodiment, the compositions provided herein can be formulated as pharmaceutical compositions. The pharmaceutical compositions can be formulated as vaccines. The vaccines can comprises the one or more attenuated or inactivated Mycobacterial strains or antigenic fragments thereof as well as one or more adjuvants. The adjuvants can be any adjuvants known in the art. In another embodiment, the compositions provided herein can be formulated as probiotics. In still another embodiment, the compositions provided herein can be formulated as postbiotics. In another embodiment, the compositions provided herein can be formulated as a food additive and/or food product. In still another embodiment, the compositions provided herein can be formulated as a nutritional supplement or dietary supplement. In yet another embodiment, the compositions provided herein can be formulated as a nutritional supplement and post-biotic. The food additive or food product may be a food or beverage, The food or beverage may be selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks

Probiotic compositions as provided herein may comprise one or more Mycobacterial strains provided herein and may be formulated such that administration of the probiotic (e.g., orally, rectally, by inhalation, etc.) results in population of the individual by the Mycobacterial strains. In some embodiments, probiotic compositions are combined and/or formulated for administration to a subject. In some embodiments, probiotics contain Mycobacterial strains at known concentrations (colony forming units or CFUs). Probiotic compositions may be in the form of a pharmaceutical-type composition (e.g., capsule, tables, liquid, aerosol, etc.) or in the form of a food supplement, additive or ingredient.

In some embodiments, probiotic microbes (e.g., Mycobacterial strains provided herein) are formulated in a pharmaceutically acceptable composition for delivery to an individual. The individual may have been diagnosed as suffering from or suspected or suffering from any malady or condition provided herein. In some embodiments, probiotics are formulated with a pharmaceutically acceptable carrier suitable for a solid or semi-solid formulation. In some embodiments, probiotic microbes are formulated with a pharmaceutically acceptable carrier suitable for a liquid or gel formulation. Probiotic formulations may be formulated for enteral delivery, e.g., oral delivery, or delivery as a suppository, but can also be formulated for parenteral delivery, e.g., vaginal delivery, inhalational delivery (e.g., oral delivery, nasal delivery, and intrapulmonary delivery), and the like.

The probiotic compositions that find use in embodiments described herein may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the probiotic microbes. In tablets, the microbes are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Other forms suitable for oral administration include liquid form preparations such as emulsions, syrups, elixirs, aqueous solutions, aqueous suspensions, or solid form preparations which are intended to be converted shortly before use to liquid form preparations. Aqueous suspensions can be prepared by dispersing the probiotic microbes in water with viscous material, such as natural or synthetic gums, resins, methylcellulose, sodium carboxymethylcellulose, and other well-known suspending agents.

The probiotic compositions (e.g., Mycobacterial strains provided herein) may be formulated for administration as suppositories. A low melting wax, such as a mixture of fatty acid glycerides or cocoa butter is first melted and the probiotic microbes are dispersed homogeneously, for example, by stirring. The molten homogeneous mixture is then poured into conveniently sized molds, allowed to cool, and to solidify.

The probiotic compositions (e.g., Mycobacterial strains provided herein) may be formulated for vaginal administration. Pessaries, tampons, creams, gels, pastes, foams or sprays, may contain agents in addition to the bacteria, such carriers, known in the art to be appropriate.

In some embodiments, probiotic compositions (e.g., Mycobacterial strains provided herein) may be formulated for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising probiotic microbes with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In some embodiments, postbiotic microbes (e.g., Mycobacterial strains provided herein that have been heat-killed or inactivated or live-attenuated) are formulated in a pharmaceutically acceptable composition for delivery to an individual. The individual may have been diagnosed as suffering from or suspected or suffering from any malady or condition provided herein. In some embodiments, postbiotics are formulated with a pharmaceutically acceptable carrier suitable for a solid or semi-solid formulation. In some embodiments, postbiotic microbes are formulated with a pharmaceutically acceptable carrier suitable for a liquid or gel formulation. Postbiotic formulations may be formulated for enteral delivery, e.g., oral delivery, or delivery as a suppository, but can also be formulated for parenteral delivery, e.g., vaginal delivery, inhalational delivery (e.g., oral delivery, nasal delivery, and intrapulmonary delivery), and the like.

The postbiotic compositions that find use in embodiments described herein may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the postbiotic microbes. In tablets, the microbes are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Other forms suitable for oral administration include liquid form preparations such as emulsions, syrups, elixirs, aqueous solutions, aqueous suspensions, or solid form preparations which are intended to be converted shortly before use to liquid form preparations. Aqueous suspensions can be prepared by dispersing the postbiotic microbes in water with viscous material, such as natural or synthetic gums, resins, methylcellulose, sodium carboxymethylcellulose, and other well-known suspending agents.

The postbiotic compositions (e.g., killed or inactivated or attenuated Mycobacterial strains provided herein) may be formulated for administration as suppositories. A low melting wax, such as a mixture of fatty acid glycerides or cocoa butter is first melted and the probiotic microbes are dispersed homogeneously, for example, by stirring. The molten homogeneous mixture is then poured into conveniently sized molds, allowed to cool, and to solidify.

The postbiotic compositions (e.g., killed or inactivated or attenuated Mycobacterial strains provided herein) may be formulated for vaginal administration. Pessaries, tampons, creams, gels, pastes, foams or sprays, may contain agents in addition to the bacteria, such carriers, known in the art to be appropriate.

In some embodiments, postbiotic compositions (e.g., killed or inactivated or attenuated Mycobacterial strains provided herein) may be formulated for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising probiotic microbes with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In some embodiments, a postbiotic can be or can be incorporated into a comestible food or beverage or ingredient thereof. The food or beverage may be selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some embodiments, a postbiotic provided herein may be a nutritional supplement. In some embodiments, a postbiotic provided herein may be a dietary supplement. In some embodiments, a postbiotic may be a selectively fermented ingredient. Postbiotics may include complex carbohydrates, amino acids, peptides, minerals, or other essential nutritional components for the survival of the bacterial composition.

Rather than pharmaceutical-type formulation, compositions provided herein may be formulated as food additive and/or food product and incorporated into a variety of foods and beverages. In one embodiment, the one or more Mycobacterium present in the composition are all non-pathogenic strains and thus the composition can be considered as safe and non-toxic for consumption by an individual. Suitable foods and beverages include, but are not limited to gums, yogurts, ice creams, cheeses, baked products such as bread, biscuits and cakes, dairy and dairy substitute foods, soy-based food products, grain-based food products, starch-based food products, confectionery products, edible oil compositions, spreads, breakfast cereals, infant formulas, juices, power drinks, and the like.

In another embodiment, administration of any composition provided herein (e.g., a composition comprising one or more Mycobacterium provided herein such as a post-biotic composition) is immunomodulatory. In other words, the one or more Mycobacterium within the compositions provided herein can have an immunomodulatory effect on an individual administered or using said composition. In this way, the one or more Mycobacterium are immunomodulators. The immunomodulatory Mycobacterium can act to alter the immune activity of a subject directly or indirectly. For example, the immunomodulatory Mycobacterium can act directly on immune cells through receptors for bacterial components (e.g. Toll-like receptors) or by producing metabolites such as immunomodulatory short chain fatty acids (SCFAs), glutathione or gamma-glutamylcysteine.

Compositions provided herein (e.g., post-biotic and/or nutritional supplements) containing immunomodulatory Mycobacterium can additionally or alternatively impact the immune activity of a subject indirectly by modulating the activity of immune cells in the subject. For example, the immunomodulatory Mycobacterium in a composition provided (e.g., post-biotic and/or nutritional supplements) may alter cytokine expression by host immune cells (e.g., macrophages, B lymphocytes, T lymphocytes, mast cells, peripheral blood mononuclear cells (PBMCs), etc.) or other types of host cells capable of cytokine secretion (e.g., endothelia cells, fibroblasts, stromal cells, etc.). In one embodiment, the host immune cell is a PBMC. In an exemplary embodiment, compositions provided herein (e.g., post-biotic and/or nutritional supplements) contain anti-inflammatory immunomodulatory bacteria that are capable of inducing secretion of anti-inflammatory cytokines by host cells. For example, anti-inflammatory bacteria can induce secretion of one or more anti-inflammatory cytokines such as but not limited to IL-10, IL-13, IL-9, IL-4, IL-5, TGFbeta, and combinations thereof, by host cells (e.g., host immune cells). In another exemplary embodiment, compositions provided herein (e.g., post-biotic and/or nutritional supplements) contain anti-inflammatory immunomodulatory Mycobacterium that are capable of reducing secretion of one or more pro-inflammatory cytokines by host cells (e.g., host immune cells). For example, anti-inflammatory Mycobacterium can reduce secretion of one or more pro-inflammatory cytokines such as but not limited to IFNgamma, IL-12p70, IL-1alpha, IL-6, IL-8, MCP1, MIP1alpha, MIP1beta, TNFalpha, and combinations thereof. Other cytokines that may be modulated by immunomodulatory Mycobacterium include, for example, IL-17A, IL-2, and IL-9. In some embodiments, the induction and/or secretion of pro-inflammatory cytokines may be induced by (e.g., in response to, either directly or indirectly) the one or more Mycobacterium present in a composition provided herein (e.g., post-biotic and/or nutritional supplements).

In some embodiments, immunomodulatory Mycobacterium are selected for inclusion in a composition of the invention (e.g., post-biotic and/or nutritional supplements) based on the desired effect of the composition on cytokine secretion by host cells, e.g., host immune cells. For example, in one embodiment, a composition (e.g., post-biotic and/or nutritional supplements) contains anti-inflammatory Mycobacterium that increase secretion of an anti-inflammatory cytokine, for example, IL-10, IL-13, IL-9, IL-4, IL-5, TGFbeta, and combinations thereof. In some embodiments, the anti-inflammatory bacteria increase secretion of two or more anti-inflammatory cytokines. In some embodiments, the anti-inflammatory bacteria increase secretion of three or more anti-inflammatory cytokines. In some embodiments, the anti-inflammatory bacteria increase secretion of four or more anti-inflammatory cytokines. In some embodiments, the anti-inflammatory bacteria increase secretion of five or more anti-inflammatory cytokines. In exemplary embodiments, the increase is an increase of at least 5%, 10%, 20%, 30%, 40%, 50%, 60%, 70%, 80%, 80%, 100%, 200%, 300%, 500% or more. In another embodiment, a composition provided herein contains anti-inflammatory Mycobacterium that decrease secretion of a pro-inflammatory cytokine, for example, IFNgamma, IL-12p70, IL-1.alpha., IL-6, IL-8, MCP1, MIP1alpha, MIPlbeta, TNFalpha, and combinations thereof. In some embodiments, the anti-inflammatory bacteria decrease secretion of two or more pro-inflammatory cytokines. In some embodiments, the anti-inflammatory bacteria decrease secretion of three or more pro-inflammatory cytokines. In some embodiments, the anti-inflammatory bacteria decrease secretion of four or more pro-inflammatory cytokines. In some embodiments, the anti-inflammatory bacteria decrease secretion of five or more pro-inflammatory cytokines. In exemplary embodiments, the decrease is a decrease of at least 5%, 10%, 20%, 30%, 40%, 50%, 60%, 70%, 80%, 80%, 100%, 200%, 300%, 500% or more. In another embodiment, the composition provided herein (e.g., post-biotic and/or nutritional supplements) contains anti-inflammatory bacteria that increase secretion of one or more anti-inflammatory cytokines and reduce secretion of one or more pro-inflammatory cytokines. Alterations in cytokine expression may occur locally, e.g., in the gastrointestinal tract of a subject, or at a site distal to the gastrointestinal tract.

In other embodiments, compositions provided herein (e.g., post-biotic and/or nutritional supplements) containing immunomodulatory bacteria impact the immune activity of a subject by promoting the differentiation and/or expansion of particular subpopulations of immune cells. For example, immunomodulatory bacteria can increase or decrease the proportion of Treg cells, Th17 cells, Th1 cells, or Th2 cells in a subject. The increase or decrease in the proportion of immune cell subpopulations may be systemic, or it may be localized to a site of action of the probiotic, e.g., in the gastrointestinal tract or at the site of a distal dysbiosis. In some embodiments, immunomodulatory Mycobacterium are selected for inclusion in a composition of the invention (e.g., post-biotic and/or nutritional supplements) based on the desired effect of the composition on the differentiation and/or expansion of subpopulations of immune cells in the subject.

In one embodiment, a composition provided herein (e.g., post-biotic and/or nutritional supplements) contains immunomodulatory bacteria that increase the proportion of Treg cells in a subject. In another embodiment, a composition provided herein contains immunomodulatory Mycobacterium that decrease the proportion of Treg cells in a subject. In one embodiment, a composition provided herein (e.g., post-biotic and/or nutritional supplements) contains immunomodulatory Mycobacterium that increase the proportion of Th17 cells in a subject (e.g., by inducing expansion of Th17 cells in the subject). In another embodiment, a composition provided herein (e.g., post-biotic and/or nutritional supplements) contains immunomodulatory Mycobacterium that decrease the proportion of Th17 cells in a subject. In one embodiment, a composition provided herein (e.g., post-biotic and/or nutritional supplements) contains immunomodulatory bacteria that increase the proportion of Th1 cells in a subject (e.g., by inducing expansion of Th1 cells in the subject). In another embodiment, a composition provided herein (e.g., post-biotic and/or nutritional supplements) contains immunomodulatory Mycobacterium that decrease the proportion of Th1 cells in a subject. In one embodiment, a composition provided herein (e.g., post-biotic and/or nutritional supplements) contains immunomodulatory Mycobacterium that increase the proportion of Th2 cells in a subject (e.g., by inducing expansion of Th2 cells in the subject). In another embodiment, a composition provided herein (e.g., post-biotic and/or nutritional supplements) contains immunomodulatory Mycobacterium that decrease the proportion of Th2 cells in a subject. The increase or decrease in the proportion of immune cell subpopulations (e.g., Th17 cells, Th1 cells and Th2 cells) may be localized or systemic.

In one embodiment, a composition provided herein (e.g., post-biotic and/or nutritional supplements) contains immunomodulatory Mycobacterium capable of modulating the proportion of one or more populations of Treg cells, Th17 cells, Th1 cells, Th2 cells, and combinations thereof in a subject. Certain immune cell profiles may be particularly desirable to treat or prevent particular disorders or maladies.

In one embodiment, use of or administration to an individual of a composition provided herein (e.g., post-biotic and/or nutritional supplements) increases immune system functioning in the individual. The increase in immune system functioning can be evidenced by the production or elevation thereof of any cytokines and/or chemokines provided herein and/or known in the art (e.g., TH1 cytokines), upregulation of granzyme B or both. The TH1 cytokines that are elevated or produced in response to administration of the compositions provided herein can include IFN-γ, IL-2, or TNF-β or a combination thereof. The increase immune system function can be evidenced by an elevation or increase in the production of one or more cytokines provided herein such as, for example, the cytokines listed in Table 1. In one embodiment, increase immune system function can be evidenced by an elevation or increase in the production of one or more cytokines selected from the group consisting of IL-6, IL-8, MIP1alpha and MIP1-beta. Assessment of the alteration in immune function can be ascertained using the methods and/or kits provided herein and/or as described in US20150301062.

TABLE 1
Non-limiting cytokine panels for use with the present invention.

| Panel 1 | Panel 2 | Panel 3 | Panel 4 | Panel 5 |
|---|---|---|---|---|
| IL5 | IFN-γ | TranSignal | Bio-Plex Pro | five or more |
| IL6 | IL-1β | Human Cytokine | magnetic | chemokines |
| IL8 | IL-2 | Antibody | Cytokine Assay | |
| IL 10 | IL-4 | Array 3.0 | (any of these | |
| IFN-γ | IL-5 | (or a subset | assays may be | |
| MCP-1 | IL-6 | of cytokines | used, i.e., the | |
| MIP-1α | IL-8 | provided in | 8-plex, 17-plex, | |
| MIP-1β | IL-10 | this assay) | 21-plex, 27-plex | |
| | TNF-α | | | |
| | MIP-1β | | | |
| | MCP-1 | | | |
| | MIP-1α | | | |
| | Rantes | | | |
| Panel 6 | Panel 7 | Panel 8 | Panel 9 | Panel 10 |
| IL5 | IL-6 | IL-2 | IL-8 | IFN-γ |
| IL6 | IL-8 | IL-4 | IL-10 | IL-1β |
| IL8 | IL-10 | IL-5 | TNF-α | IL-2 |
| IL 10 | TNF-α | IL-6 | MIP-1β | |
| IFN-γ | MIP-1β | | MCP-1 | |
| | MCP-1 | | Rantes | |
| | MIP-1α | | | |

### Administration of Compositions

In some embodiments, a composition (e.g., postbiotic composition and/or food product or additive or supplement) provided herein is administered over a dosing time period (e.g., <1 minute, <1 hour, <2 hours, <4 hours, <6 hours, <12 hours, <24 hours, etc.) in an amount that is sufficient to provide a desired therapeutic benefit (e.g., modulation of immune system functioning of an individual administered or using the composition). In some embodiments, the dose of the composition provided herein (e.g., post-biotic and/or nutritional supplements) is administered for the dosing time period in a concentration of from about 10 to about 1×10¹⁴ colony forming units (cfus) of the one or Mycobacterial strain(s) (e.g., 10 cfu, 100 cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, or any suitable ranges therein (e.g., from about 10² cfu to about 10¹³ cfu, about 1×10⁴ to about 1×10¹¹ cfu, about 1×10⁶ to about 1×10⁹ cfu, about 1×10¹⁰ to about 1×10¹² cfu, etc.), etc.). In some embodiments, the dose of the composition provided herein (e.g., post-biotic and/or nutritional supplements) is administered for the dosing time period in a concentration of about 1×10¹⁰ cfus, 5×10⁹ cfus, 2.5×10⁹ cfus, 2×10⁹ cfus, 1×10⁹ cfus, 5×10⁸ cfus, 2.5×10⁸ cfus, 2×10⁸ cfus, 1×10⁸ cfus, 5×10⁷ cfus, 2.5 10⁷ cfus, 2×10⁷ cfus, 1×10⁷ cfus, 5×10⁶ cfus, 2.5×10⁶ cfus, 2×10⁶ cfus, 1×10⁶ cfus, 5×10⁵ cfus, 2.5×10⁵ cfus, 2×10⁵ cfus, 1×10⁵ cfus, 5×10⁴ cfus, 2.5×10⁴ cfus, 2×10⁴ cfus or 1×10⁴ cfus of the one or Mycobacterial strain(s). In some embodiments, the dose of the composition provided herein (e.g., post-biotic and/or nutritional supplements) is administered for the dosing time period in a concentration of about 250 mg, 100 mg, 10 mg or 1 mg of the one or Mycobacterial strain(s). In some cases, there are 5 billion (i.e., 5×10⁹ cfus) in a 250 mg dose. In some cases, there are 2 billion (i.e., 2×10⁹ cfus) in a 100 mg dose. In some cases, there are 2×10⁸ cfus in a 10 mg dose. In some cases, there are 2×10⁷ cfus in a 1 mg dose.

The compositions of the invention may be administered to mammals (e.g., rodents, humans) in any suitable formulation. For example, isolated Mycobacterium or antigenic fragments thereof may be formulated in pharmaceutically acceptable carriers or diluents such as physiological saline or a buffered salt solution. Suitable carriers and diluents as provided herein can be selected on the basis of mode and route of administration and standard pharmaceutical practice.

The compositions of the invention may be administered to mammals by any conventional technique. Typically, such administration will be oral, sublingual, nasal, pulmonary or parenteral (e.g., intravenous, subcutaneous, intravesicular, intramuscular, intraperitoneal, intradermal, subdermal, or intrathecal introduction). The compositions may also be administered directly to a target site by, for example, surgical delivery to an internal or external target site, or by catheter to a site accessible by a blood vessel. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Dosing

The compositions (e.g., food product formulation comprising one or more Mycobacterium as provided herein) are preferably administered to a mammal (e.g., a human) in an effective amount, that is, an amount capable of producing a desirable result in an individual administered the composition (e.g., activating or boosting the immune response). Such an effective amount can be determined as described below.

As is well known in the medical and veterinary arts, dosage for any one subject depends on many factors, including the subject's size, body surface area, age, the particular composition to be administered, time and route of administration, general health, and other drugs being administered concurrently.

In certain embodiments, a particular dosage of a composition provided herein is administered to or consumed by a subject. In certain embodiments of the invention, there is provided a composition comprising a live-attenuated or heat-killed bacteria for use in the present invention, which typically may be from 10³ to 10¹¹ cells or colony forming units (CFUs), from 10⁴ to 10¹⁰ cells or CFUs, from 10⁶ to 10¹⁰ cells or CFUs, or 10⁶ to 10⁹ cells or CFUs per unit dose. The effective amount of live-attenuated or heat-killed Mycobacterium for use in the methods or compositions provided herein can be from 10³ to 10¹¹ cells or CFUs, from 10⁴ to 10¹⁰ cells or CFUs, from 10⁶ to 10¹⁰ cells or CFUs, and from 10⁶ to 10⁹ cells or CFUs per unit dose. The unit dose can be 5 ul, 10 ul, 20 ul, 30 ul, 40 ul, 50 ul, 60 ul, 70 ul, 80 ul, 90 ul, 100 ul, 125 ul, 150 ul, 175 ul, 200 ul, 250 ul, 300 ul, 350 ul, 400 ul, 450 ul, 500 ul, 600 ul, 650 ul, 700 ul, 750 ul, 800 ul, 850 ul, 900 ul, 950 ul, 1000 ul or 1500 ul. In one embodiment, the composition comprises an effective amount of live-attenuated or heat-killed Mycobacterium is from 1.8×10⁶ to 3.9×10⁶ colony forming units per unit dose. In one embodiment, the composition comprises an effective amount of live-attenuated or heat-killed Mycobacterium is from 1×10⁹ to 10×10⁹ colony forming units per unit dose. In one embodiment, the composition comprises an effective amount of live-attenuated or heat-killed Mycobacterium is from 2×10⁹ to 5×10⁹ colony forming units per unit dose. In one embodiment, the composition comprises an effective amount of live-attenuated or heat-killed Mycobacterium is from 2×10⁸ to 5×10⁹ colony forming units per unit dose. In one embodiment, the composition comprises an effective amount of live-attenuated or heat-killed Mycobacterium is from 2×10⁷ to 5×10⁹ colony forming units per unit dose. In one embodiment, the composition comprises an effective amount of live-attenuated or heat-killed Mycobacterium is from 1×10⁶ to 5×10⁶ colony forming units per unit dose. In one embodiment, the composition comprises an effective amount of live-attenuated or heat-killed Mycobacterium is from 1×10⁵ to 1×10⁶ colony forming units per unit dose. In one embodiment, the composition comprises an effective amount of live-attenuated or heat-killed Mycobacterium is from 1×10⁴ to 1×10⁵ colony forming units per unit dose. Alternatively, the dose of a composition provided herein can be from 0.01 mg to 1 mg, 0.1 mg to 0.5 mg, 0.5 mg to 1 mg, 1 mg to 1.5 mg, 1.5 mg to 2.0 mg, 2 mg to 5 mg, 5 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 150 mg, 150 mg to 200 mg or 200 mg to 250 mg. In one embodiment, the dose is 1 mg. In one embodiment, the dose is 0.5 mg. In one embodiment, the dose is 0.1 mg. In one embodiment, the dose is 1 mg. In one embodiment, the dose is 10 mg. In one embodiment, the dose is 100 mg. In one embodiment, the dose is 250 mg. In one embodiment, the dose is 250 mg and comprises 5×10⁹ cfus. In one embodiment, the dose is 100 mg and comprises 2×10⁹ cfus. In one embodiment, the dose is 10 mg and comprises 2×10⁸ cfus. In one embodiment, the dose is 1 mg and comprises 2×10⁷ cfus. The organisms or antigenic fragments derived therefrom can be presented as either a suspension or dry preparation.

The composition according to the invention may also comprise, such as pharmaceutically acceptable additives, e.g. solvents, adjuvants, carriers and/or preservatives as provided herein.

The methods provided herein can be conducted as a series of administrations with increasing doses during a specific period. In one example, the composition can be administered or consumed in 8-10 increasing doses during 4-12 weeks, preferably 8-10 weeks. In one embodiment, the composition can be administered or consumed in 2 doses spaced 4 weeks apart.

In order to obtain the desired effect for a prolonged period of time a composition provided herein may be administered or consumed at several occasions. For example, a first series may be followed by repeated administrations or uses given at specified intervals. The specified intervals can be approximately once a week for 5-15 weeks, preferably for 10 weeks.

These repeated administrations can result in an unspecific or specific activation of the immune system over a long period of time.

### Assessing Immune System Modulation

In another aspect, the invention provides methods for modulating the immune system of an individual by administering a composition as provided herein that comprises one or more Mycobacterium. The Mycobacterium can be a non-pathogenic strain as provided herein. The individual may be suffering from or suspected of suffering from one or more diseases or disorders as provided herein. The method for modulating an individual's immune system involves determining or detecting as a baseline the level of one or more cytokines expressed in the individual prior to administration or use of a composition provided herein. Following administration or use of a composition provided herein, subsequent measurements of one or more cytokine levels are carried out to determine the levels or patterns of expression of the one or more cytokines. The altered levels and/or patterns of expression of one or more of the cytokines measured in the individual undergoing administration or use of a composition provided herein are compared to the levels or patterns of expression of cytokines in a control. In one embodiment, the control is the levels and/or patterns of expression of the one or more cytokines in the individual before administration or use of a composition provided herein. In another embodiment, the control is the levels and/or expression levels of the one or more cytokines from a healthy patient, or cytokine levels reported for a patient without a malady suffered by an individual who is administered or is using a composition provided herein. In another embodiment, the control is the levels and/or expression levels of the one or more cytokines from a patient with the same malady suffered by the individual who is administered or is using a composition provided herein, but who is not administered or using a composition as provided herein.

In one embodiment, the methods for assessing immune system modulation involve determining or assaying the levels of at least one, at least two, at least three, at least four, at least five, at least six, at least seven, at least eight, at least nine, or at least ten or more cytokines in the plasma of blood samples obtained from individuals after use of the compositions (e.g., the compositions comprising Mycobacterium as provided herein) and comparing the levels of the assayed cytokines to a control. The control can be any control as provided herein. The control can be a healthy patient, a patient without a malady suffered by an individual who is administered or is using a composition provided herein, a patient with the same malady suffered by the individual who is administered or is using a composition provided herein, but who is not administered or using a composition as provided herein or the individual before administration or use of a composition provided herein. In a further embodiment, the method involves determining or assaying the levels of at least one, at least two, at least three, at least four, at least five, at least six, at least seven, at least eight, at least nine, or at least ten or more cytokines in the peripheral blood mononuclear cells (PBMCs) that have been separated from the plasma of blood samples obtained from the individuals after treatment and comparing the levels of the assayed cytokines to a control. The control can be any control as provided herein. The control can be a healthy patient, a patient without a malady suffered by an individual who is administered or is using a composition provided herein, a patient with the same malady suffered by the individual who is administered or is using a composition provided herein, but who is not administered or using a composition as provided herein or the individual before administration or use of a composition provided herein. These levels are then analyzed to determine if the levels are altered due to the treatment. For example, the levels in the individual's sample during and/or after treatment, in one embodiment, are compared to levels in a control sample, for example, a sample known to not have a malady suffered from the individual. In another embodiment, control levels are known, for example, from a database. In one embodiment, a change in expression in a majority of the cytokines tested toward the levels in the control is determinative/indicative of the administration or use of the composition being efficacious. In another embodiment, a change in expression of at least about 33% or at least about 67% of the cytokines tested is determinative/indicative of an effective immune system modulation. In a further embodiment, a treatment with a composition as provided herein is deemed to be efficacious if at least about 75%, or at least about 75% or more of the cytokines tested have altered expression due to use of a composition provided herein. In even a further embodiment, use of a composition as provided herein is deemed to be efficacious if the expression level of every cytokine tested, or about every cytokine tested in the patient is altered. The altered expression of one or more cytokines during or following use can be modifying the level or expression of the one or more cytokines to be substantially the same expression level of the one or more cytokines in a control as provided herein. As used herein, the term “substantially the same expression level” can be about 55%, about 60%, about 65%, about 70%, about 75%, about 80%, about 85%, about 90%, about 95%, about 99% or about 99% of the expression level of a particular cytokine in a control as provided herein.

The present invention is not limited by any particular combination of cytokines. For example, the cytokines whose expression can be evaluated in order to determine immune system modulation can be selected from IL-1, IL-2, IL-4, IL-5, IL-6, IL-7, IL-8, IL-10, IL-12, IL-13, IL-15, IL-17, IL-21, IFN-7, IFN-α, TNF-α, IP-10, MCP-1, MIG, MIP-1α, MIP-1β, GM-CSF, Eotaxin, RANTES, etc. or a combination thereof. In another aspect, the invention further includes determining the levels of one or more of IL-lRA, TL2R, IL-7, IL-12 (p40/p70), IL-13, IL-15, IL-17, IFN-α, IP-10, MIG, VEGF, G-CSF, EGF, granzyme B, FGF-basic and HGF or a combination thereof. In yet another aspect, the invention also includes determining the levels of IL-9 and PDGF-BB or a combination thereof. In one embodiment, the cytokines whose expression is evaluated in order to determine immune system modulation are selected from the group consisting of IL-6, IL-8, MIP-la, MIP-10 and any combination thereof. The cytokine may be inflammatory or anti-inflammatory. In one embodiment, the cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of cytokine assayed maybe a mRNA, miRNA, or DNA. In another example, the expression levels of cytokines included in commercial cytokine panels (or cytokine subsets thereof) can be evaluated by the methods provided herein. Various combinations of cytokines for use in the present invention are provided in the Table 1 as provided herein. Subsets of these combinations may also be used in the methods provided herein. It should be understood that these combinations are representative, and should not be construed as limiting the invention.

### Measurement/Detection of Cytokine Levels

In one embodiment, cytokine levels in methods entailing modulating (e.g., increasing) immune system efficiency as provided herein are tested on the protein level. In another embodiment, cytokine levels in methods entailing modulating (e.g., increasing) immune system efficiency as provided herein are determined at the mRNA level. In yet another embodiment, both mRNA and protein levels for the cytokines are examined in the methods provided herein. Methods for assaying cytokines at the protein or mRNA levels are well known in the art and can be employed in the methods provided herein.

Measuring cytokine levels in methods entailing modulating (e.g., increasing) immune system efficiency as provided herein can be from blood or a plasma sample that may be stimulated or un-stimulated. That is, cell proliferation may be induced prior to assaying the cytokine levels. In one embodiment, the PBMCs are un-stimulated. In another embodiment, the PBMCs are stimulated to cause proliferation of the cells prior to assaying for cytokines. Methods for stimulating PBMCs are known in the art, and include, but are not limited to, the addition of mitogens to the cells. Non-limiting examples of mitogens include lipopolysaccharide (LPS), phytohemagglutinin (PHA), or phorbol ester, such as phorbol myristate acetate (PMA) with or without ionomycin, pokeweed mitogen (PWM), concavalin A (Con-A), or combinations thereof.

In one embodiment, cytokine expression is measured at the mRNA level, for example, by quantitative RT-PCR (also known as real time RT-PCR). mRNA expression levels can also be measured by Northern blot assay, array hybridization, sequencing, etc. For example, multiplex quantitative RT-PCR, in one embodiment, is carried out to determine the mRNA expression levels of a cytokine panel. Cytokine RT-PCR kits are commercially available, for example, from Roche.

In another embodiment, secreted cytokine levels are determined (i.e., at the protein level). In one embodiment, secreted cytokine levels are determined by using an antibody array, for example, the TranSignal Human Cytokine Antibody Array 3.0, available from Panomics. The Panomics array includes antibodies directed to the following cytokines: Apol/Fas, Leptin, Rantes, ICAM-1, IL-2, IL-7, CTLA, MIP-la, MIP-10, TGFβ, VCAM-1, IL-3, IL-8, IL-4, IL-10, IL-5, IL-12, IL-6, IL-15, IL-6R, IL-17, IL-1Ra, IL-10, IL-la, VEGF, IFNγ, TNFα, TNFRI, TNFRII, MIP-5, MIP-4, MMP3, Eotaxin, GM-CSF, EGF, IP-10. In this embodiment, not all cytokines in the array need be probed for. For example, the expression levels of a subset of five cytokines, or five or more cytokines, or six cytokines, or six or more cytokines, or seven cytokines, or seven or more cytokines, or ten cytokines, or ten or more cytokines, or twelve cytokines, or twelve or more cytokines may be determined when carrying out the methods of the invention.

Secreted cytokine levels, in one embodiment, are determined with a multiplex immunoassay built on magnetic beads. For example, in one embodiment, the Bio-Plex Pro magnetic Cytokine Assay is used (Bio-Rad). In this embodiment, the Assay is commercially available as a ready to use kit, for example, for the detection of eight cytokines, seventeen cytokines, 21 cytokines or 27 cytokines. The full number or a subset of the cytokines may be detected in the methods of the invention. Alternatively, expression levels of cytokines can be tested in a sample by doing multiple assays on the sample, for example, in “singleplex” format. In one embodiment, the Bio-Rad singleplex cytokine assays are used.

Another antibody based bead assay is available from Invitrogen, and is also amenable to be used in the methods of the present invention. Specifically, the Human Cytokine Thirty-Plex antibody bead kit may be employed to detect the levels of a panel of cytokines in an individual. Although the assay can detect the levels of thirty cytokines, not all thirty need to be detected in order to carry out the methods provided herein. For example, as provided above, five, six, seven, eight, nine, ten, eleven or twelve cytokines can be assayed for their expression levels. The Invitrogen kit comprises analyte specific components for the measurement of human IL-1β, IL-1RA, IL-2, IL-2R, IL-4, IL-5, IL-6, IL-7, IL-8, IL-10, IL-12p40/p70, IL-13, IL-15, IL-17, TNF-α, IFN-α, IFN-7, GM-CSF, MIP-1α, MIP-1β, IP-10, MIG, Eotaxin, RANTES, MCP-1, VEGF, G-CSF, EGF, FGF-basic, and HGF. These reagents, in one embodiment, are used in the Luminex® 100™ or 200™ System.

Methods for assaying cytokines at the protein or mRNA levels are well known in the art. Besides the assays provided above, other non-limiting examples of methods for assaying cytokines at the protein level include enzyme-linked immunoassay (ELISA), Tetramer assay, ELISPOT assay, Fluorospot assay, etc. The cytokines concentration in the plasma, culture supernatant, or cell lysate derived from PBMC can be measured, for example, by multiplex immunoassay based on Luminex xMAP bead array technology, or Bio-Plex 200 fluorescence bead reader (BioRad Laboratories, Hercules, CA). In one embodiment, the level of one or more cytokine mRNA can be detected (measured) by real time PCR, RT-PCR, Northern blot assay, array hybridization, sequencing, etc. The altered level(s) of the cytokines measured in the affected individual compared to the level from control group is predictive/indicative of a modulation (e.g., increase) in immune system functioning in the individual. As provided herein the individual whose immune system is modulated following use or administration or use of a composition provided herein suffers from one or more of the diseases or disorders provided herein. The cytokine levels in the individual, in one embodiment, can be higher than the cytokine levels of a healthy patient, for each cytokine tested. The cytokine levels for each cytokine tested in the individual, in one embodiment, can be higher than the cytokine levels of a patient suffering from the same one or more diseases or disorders of the individual, but who has not been administered or used a composition provided herein.

The level of cytokines can be determined using an algorithm and the raw data obtained by measuring the levels of cytokines which have been stored in a computer system, or any other medium that is linked to a computer or machine. In one aspect, the method further includes evaluation of the individual's (i.e., individuals diagnosed with a disease or disorder provided herein) clinical and physical symptoms as described herein in conjunction with determining the levels of one or more cytokines.

As it relates to the methods provided herein, cytokine expression can be “altered” or “differentially expressed”, in an individual, in one embodiment, if expression of the cytokine in the individual's sample is at least about 1.5 times higher or lower than the expression of the same cytokine in a control. In another embodiment, cytokine expression is “altered” if cytokine expression in the individual's sample is at least about 2 times higher or lower than the expression of the same cytokine in a control (e.g., levels reported for a healthy patient or patient not administered or used a composition provided herein). In another embodiment, cytokine expression is “altered” if cytokine expression in the individual is at least about 2.5 times higher or lower (or at least about 2.5 times or more higher or lower) than the expression level of the same cytokine in a control. In yet another embodiment, cytokine expression is “altered” if cytokine expression in the individual is at least about 3 times higher or lower (or at least about 3 times or more higher or lower) than the expression level of the same cytokine in a control. In another embodiment, cytokine expression is “altered” if cytokine expression in the individual is at least about 5 times higher or lower (or at least about 5 times or more higher or lower) than the expression level of the same cytokine in a control. In even another embodiment, cytokine expression is “altered” if cytokine expression in the individual is at least about 10 times higher or lower than the expression level of the same cytokine in a control. In yet another embodiment, cytokine expression is altered if cytokine expression in the individual is at least about 10 times or more, higher or lower, than the expression level of the same cytokine in a control. As provided above, the control may be from a non-disease subject or an individual with the same malady, but not administered or used a composition provided herein.

Altered expression of the cytokine may be the same or different for each individual cytokine that is differentially expressed. For example, the expression of one cytokine (mRNA or protein) may be 2× lower, or about 2× lower, than the expression of the same cytokine in a control sample, while the expression of a second cytokine may be 1.5× lower, or about 1.5× lower, than the expression of the same cytokine in a control sample. As discussed above, altered expression includes both higher and lower expression of the cytokine, compared to a control level.

### EXAMPLES

The present invention is further illustrated by reference to the following Examples. However, it should be noted that these Examples, like the embodiments described above, are illustrative and are not to be construed as restricting the scope of the invention in any way.

### Example 1: Examination of Immune Modulating Effects of Mycobacteria

### Introduction and Objective

Several species of Mycobacteria have been identified as having the ability to modulate immune responses, even as heat-killed preparations (see Bazzi et al., Innate Immunity 2017, vol 23(7), 592-605; Bach-Griera et al., Vaccines 2020, 8, 198; Liu et al., Curr Microbiol (2015) 71:129-135). Applications of these bacteria have been utilized for immunotherapy in the treatment of multiple types of cancer (see Noguera-Ortega et al., European Urology Focus 2(2016) 67-76; Morales, Alvaro, The Canadian Journal Of Urology; 24(3); June 2017; Yuksel et al., J Mol Microbiol Biotechnol 2011; 20: 24-28) as well as having molecular effects on intestinal and extra-intestinal organs, including but not limited to microbiome interactions and in reference to immune-mediated diseases. Immune modulating Mycobacteria have been especially detailed in reference to the use of Mycobacterium bovis (M. bovis) as a consequence of the utilization of the BCG vaccine. However, side effects of the BCG vaccine have been well-documented (Witjes J A et al., Eur Urol Suppl 2008; 7(10): 667-674).

The primary objective of this Example was to identify Mycobacteria that could potentially act in a safe and non-toxic immune modulating manner by promoting the production of specific chemokine and cytokine responses with a potential application for impacting the microbiome. The following Mycobacteria strains were used: Mycobacterium agri (M. agri), Mycobacterium phlei (M. phlei), Mycobacterium tokaiense (M. tokaiense), Mycobacterium smegmatis (M. smegmatis), Mycobacterium brumae (M. brumae), Mycobacterium aurum (M. aurum), and Mycobacterium obuense (M. obuense) and their potential immune modulating effects were compared to the reaction to Bacillus subtilis (B. subtilis) and PHA.

### Materials and Methods

Ethics Statement

Informed signed written consent for blood collection and testing was obtained from healthy volunteers using a form and procedures approved by the Institutional Review Board Services, Aurora, Ontario, Canada.

### Bacterial Cells

For immunologic challenges Mycobacteria strains, M. agri, ATCC27406; M. phlei, ATCC11758; M. tokaiense, ATCC27282; M. smegmatis, ATCC19420; M. brumae, ATCC51384; M. aurum, ATCC23366; M. obuense, ATCC27023; as well as B. subtilis, ATCC6051 were purchased from the American Tissue Culture Collection (ATCC). M. Smegmatis isolates were provided by the Institute for Tuberculosis Research, College of Medicine at the University of Illinois at Chicago. Mycobacteria strains were grown in the media shown in Table 2. The media composition had the pH adjusted to 7.4 and was filter sterilized (filter pore size: 0.22 microns). A loopful of each strain was inoculated separately into 15 mL of media in 50 mL bioreaction tubes and incubated at 37° C. wish shaking (220 rpm) for 3 days. A medium control (medium without inoculum) was also incubated along with the Mycobacterium samples as a blank control. After 3 days, cells were collected by centrifugation (9000 rpm) and washed three times with borate buffered saline pH 8.0, weighed (wet weight) and resuspended in the same buffer to make 100 mg/mL. The cells were heat-killed by autoclaving (i.e., autoclaved at 121° C. for 20 minutes). The samples were also submitted for mass spectrometry (MS) analysis and the results showed no media residue in the samples.

TABLE 2
Media composition used for growing Mycobacterium.

| Component | Quantity | |
|---|---|---|
| L-asparagine | 5 | g/L |
| Potassium dihydrogen phosphate | 5 | g/L |
| Citric acid | 1.5 | g/L |
| Magnesium sulphate | 0.5 | g/L |
| Glycerol | 20 | mL/L |
| Tween 80 | 0.2% | v/v |

The dry-freezed B. subtilis samples were prepared by washing bacterial pellets with borate-buffered saline, pH 8.0 three times, weighed and suspended in phosphate-buffered saline, pH 7.4 at 100 mg/ml (wet weight). The samples were then autoclaved as described above.

PBMC Challenges

Peripheral blood mononuclear cells (PBMC) were isolated from blood collected from healthy individuals by density gradient centrifugation as described earlier (Behm et al., 2012). Cells were cultured at 10⁶ cells/ml in RPMI 1640 Medium supplemented with 0.5% penicillin-streptomycin solution (10,000 U/ml penicillin, 10,000 μg/ml streptomycin) 0.5% L-glutamine and 5% fetal bovine serum. One ml PBMC cultures were placed in 24-well tissue culture dishes followed by the addition of heat-killed bacterial cells. Bacterial cells were added at various concentrations from 50 μg/ml to 500 μg/ml as indicated below. Ten pg/ml phytohemagglutinin (PHA-P, Sigma-Aldrich, St. Louis, MO) was added to separate wells as a positive control. Negative control samples contained media only and were used for determining the basal levels of cytokine secretion. Plates were placed in a carbon dioxide water jacketed incubator and incubated for 18 hours.

Cytokine Assay

After overnight culture of PBMC, 0.5 ml supernatant was removed from each plate, centrifuged at 16,000 g at +4 degree Centigrade for 2 minutes and clear cell-free liquid was kept frozen at −70 C. Cytokine and chemokine concentrations in PBMC tissue culture supernatants were measured via multiplex immunoassay based Luminex xMAP bead array technology using a Luminex MagPix fluorescence bead reader (Wallace et al., 2015). Four regions of antibody-conjugated beads were used for measuring human cytokines IL-6, IL-8, as well as chemokines MIP-1 alpha and MIP-1 beta.

Statistical Methods

Z-test was used to compare cytokine and chemokine responses in immune challenges. The confidence level in all tests was set at 5%. Correlations between productions of different cytokines and chemokines in immune challenges were evaluated by using the Pearson correlation test. For this test, normalized cytokine concentration values were used to account for the large differences in the levels of individual cytokines and chemokines. The correlation of cytokine and chemokine productions in two challenges was considered strong when their r value was larger than 0.7. All statistical analyses were performed in Microsoft Excel.

### Results and Discussion

M. smegmatis Stimulated Extracellular Productions of Cytokines and Chemokines in PBMC Cultures

Three isolates of M. smegmatis strains collected from feline skin, abdomen, and an abscess were characterized, grown and maintained at the Institute for Tuberculosis Research, College of Medicine at the University of Illinois at Chicago. To determine immune responses to these isolates, PBMCs were isolated from healthy individuals and cultured in the presence of either 50 μg/ml, 100 μg/ml or 200 μg/ml of heat-killed bacteria. Subsequently, the concentrations of cytokines IL-6, IL-8, and chemokines MIP-1alpha and MIP-1beta were measured in the tissue culture supernatants. As shown in FIGS. 1A-1D, all three isolates stimulated secretions of the four cytokines/chemokines showing a significant increase in cytokine and chemokine levels in stimulated PBMC cultures compared to controls. Protein levels did not increase at higher mycobacterial cell concentrations, which suggested that the immune responses were close to saturation levels.

To determine if all three isolates had the same effect on the immune cells, a correlation analysis of extracellular productions of different cytokines and chemokines in these challenges was performed. The Pearson correlation test was performed by using the concentrations of four cytokines/chemokines normalized to mean concentration values across all challenges to correct for large differences in levels of the different cytokines and chemokines (FIGS. 1A-1D). As shown in Table 3, secretions of all cytokines and chemokines correlated well for all three M. smegmatis challenges suggesting that all three isolates evoked the same immune response. This also provided indirect evidence that these mycobacterial preparations were indeed from the same strain. In addition, Mycobacterium-specific immune responses was also compared to responses to the mitogenic activator phytohemagglutinin (PHA, Behm et al., 2012). The Pearson correlation test showed no or negative correlations between cytokine and chemokine levels in the PHA challenge compared to any M. smegmatis challenge (Table 3), which suggested that cytokine and chemokine production patterns were distinct in the PHA challenge.

TABLE 3
Spearman correlation coefficients of cytokine and chemokine
expressions in challenges to M. smegmatis isolates

| | Cell concentration | | | | | | | | |
|---|---|---|---|---|---|---|---|---|---|
| | 50 μg/ml | | | 100 μg/ml | | | 200 μg/ml | | |
| Challenge | FB | FA | FS | FB | FA | FS | FB | FA | FS |
| FB | | | | | | | | | |
| FA | 0.75 | | | 0.77 | | | 0.98 | | |
| FS | 0.86 | 0.95 | | 0.84 | 0.99 | | 0.98 | 0.92 | |
| PHA | −0.51 | 0.01 | −0.03 | −0.80 | −1.00 | −1.00 | −0.60 | −0.61 | −0.60 |

M. smegmatis and M. agri Activated the Same Cytokine and Chemokine Expression Pathway in PBMC Challenges

To compare cellular immune responses to M. smegmatis with responses to other Mycobacterium strains, PBMCs were challenged to either 50 μg/ml, or 100 μg/ml of mycobacterial preparations from the following strains: M. agri, M. phlei, M. tokaiense, M brumae, M. aurum, and M. obuense. M. smegmatis type strain was used as a reference. As shown in FIGS. 2A-2D, apart from M. smegmatis, M. agri induced high immune responses in mononuclear cells manifested by significant extracellular cytokine and chemokine productions. The concentrations of all four cytokines/chemokines (IL-6, IL-8, MIP-la and MIP-10) were higher at 100 μg/ml of these Mycobacterium strains vs. the lower doses. Some increases in cytokine and chemokine levels were also observed in response to 50 μg/ml M brumae. However, concentrations of these proteins decreased at 100 μg/ml M. brumae. Also, very weak responses were observed in the M. obuense challenges. Significant patient-to-patient variations were observed in cytokine and chemokine responses (compare FIGS. 2A-2D to FIGS. 1A-1D). The Pearson test showed a strong correlation of cytokine and chemokine productions for M. smegmatis and M. agri challenges (r=0.93, Table 4). Again, no positive correlations were observed for productions of different proteins in PHA challenge compared to either mycobacteria challenge (Table 4). Similar patterns of extracellular cytokine and chemokine productions in these challenges suggested that expressions of the cytokines IL-6, IL-8, and the chemokines MIP-la and MIP-10 were activated by a similar mechanism. In contrast, distinct cellular responses to PHA indicated that PHA activated different or distinct cytokine and chemokine response pathways.

To further confirm that M. smegmatis and M. agri activated the same cytokine and chemokine expression pathway in immune cells, PBMCs were challenged to the mixture of M. smegmatis and M. agri at 50 μg/ml each and compared cytokine and chemokine concentrations in the mixed challenge to their levels in individual challenges to 100 μg/ml bacterial cells. It was thought that if both mycobacteria strains evoked the same immune response, exposures to the mixture of two strains would elevate the level of each protein to the value, which would be the average of two protein concentrations in individual challenges. Indeed, as shown in FIGS. 3A-3D, for all four analytes, each protein concentration in PBMC cultures challenged to the mixture of two Mycobacterium strains was close to the average of two concentrations in individual challenges. The Z-test did not show any statistically significant differences between these two values for all four cytokines/chemokines. The observation that challenges to M. smegmatis and M. agri produced similar secretion patterns for different cytokines and chemokines suggested that these Mycobacterium strains activated immune cells by the same mechanism.

PBMC Responses to Mycobacterium Strains were Distinct from Responses to B. subtilis

Immune responses to M. smegmatis and M. agri to responses were also compared to other bacteria of distant classification lineages. B. subtilis, is a Gram-positive bacteria which belongs to the phylum Firmicutes. In contrast, the Mycobacterium genus, contains Gram-negative bacteria species from the distant Actinobacteria phylum. Previous studies showed that B. subtilis activated immune responses in vivo (Freedman et al., 2021) and induced cytokine productions in PBMC cultures (Shakh et al., 2012). To compare cellular responses to B. subtilis with responses to Mycobacterium strains, PBMC cultures were challenged with various concentrations of heat-killed B. subtilis cell preparations and the cytokine and chemokine levels in the stimulated cultures were measured. As shown in FIGS. 4A-4B, B. subtilis preparations at 100 μg/ml (BS20 in Table 4) induced productions of the cytokines IL-6, IL-8, and the chemokines MIP-la and MIP-10 at high levels. When cellular immune responses in B. subtilis challenges were compared with either M. smegmatis (MS20, 100 μg/ml), M. agri (MA20, 100 μg/ml) or PHA challenges, it appeared that cytokine and chemokine productions in the B. subtilis challenge did not correlate with their levels in other challenges (Table 4). These results suggested that cellular responses to B. subtilis were distinct from responses to Mycobacterium strains or PHA.

TABLE 4
Spearman correlation coefficients of cytokine
and chemokine expression in PBMC challenges.

| Challenge | MS20 | MA20 | BS20 | PHA |
|---|---|---|---|---|
| MS20 | 1 | | | |
| MA20 | 0.93 | 1 | | |
| BS20 | −0.32 | −0.29 | 1 | |
| PHA | −0.93 | −0.95 | 0.02 | 1 |

Since challenges to B. subtilis resulted in distinct patterns of cytokine and chemokine productions, this strain may have engaged a different activation mechanism of cytokine and chemokine expressions than Mycobacterium strains. In this case, a combined Mycobacterium and B. subtilis challenge would have an additive effect on cytokine and chemokine levels. To test this hypothesis cytokine and chemokine concentrations in PBMC cultures challenged to the mixture of 50 μg/ml B. subtilis and 50 μg/ml Mycobacterium preparations were compared with protein levels in challenges to 100 μg/ml bacterial preparations of either strain alone. Again, because higher concentrations of bacterial cells tend to suppress protein secretion (see FIGS. 1A-1D), the “average” effect would indicate that these two strains activate the same cytokine and chemokine production pathway, while higher than average protein levels would indicate two different activation mechanisms. As shown in FIGS. 5A-5D, the cultures challenged to the mixtures of B. subtilis and any Mycobacterium strain produced significantly higher IL-6 concentrations than the averages of two IL-6 concentrations in individual challenges (FIG. 5A). However, significant differences in IL-8 and MIP-10 levels were observed only in the combined B. subtilis and M. agri challenge (FIG. 5B and FIG. 5D). Also, differences in MIP-la levels for mixed challenges were not significant, which may be attributed to a higher variation in measuring concentrations of this cytokine (FIG. 4C). These results demonstrated that cellular responses to B. subtilis and Mycobacterium strains were different and their combination had higher than average effects on cytokine and chemokine production. It was concluded that challenges to B. subtilis and Mycobacterium strains engaged different immune response pathways, thereby affecting their combined effects on extracellular cytokine and chemokine production.

Weak PBMC Responses to Other Mycobacterium Strains were not Due to Cellular Toxicity or Immunosuppression

Significant PBMC responses to M. phlei, M. tokaiense, M. aurum and M obuense (FIGS. 2A-2D) were not detected. Decreases in cytokine and chemokine production at higher concentrations of M. smegmatis isolates (FIGS. 1A-1D) and M. brumae (FIGS. 2A-2D) were also observed. This observation raised the possibility of cellular toxicity and/or immune suppression induced by these bacterial preparations. To confirm or rule out this possibility, 50 μg/ml Mycobacterium strains was added to 50 μg/ml B. subtilis in PBMC challenges and determined if Mycobacterium cell preparations suppressed immune responses to B. subtilis. As shown in FIGS. 5A-5D, addition of any Mycobacterium preparations to B. subtilis in PBMC challenges did not inhibit cytokine and chemokine productions. Significant increases in protein levels in response to the M. brumae mixture were also observed, consistent with elevated responses to this Mycobacterium strain (FIGS. 2A-2D). Concentrations of IL-6 and MIP-la also increased for the M. phlei mixture (FIGS. 5A-5B) and a significant elevation of IL-6 levels was observed when M. tokaiense was added to the challenge (FIG. 5A). There was a slight statistically significant decrease in IL-8 concentrations for the M. obuense mixture (FIG. 5C), consistent with the suppression of the immune response seen at the higher concentration of this strain (FIGS. 2A-2D). It was therefore concluded that that the lack of immune activities of Mycobacterium strains in PBMC challenges was not due to the concomitant cellular toxicity or immunosuppression induced by these bacterial preparations at concentrations tested.

### CONCLUSIONS

Identification of potential immune-modulating properties of multiple Mycobacterium species in a heat-killed format was sought in an effort to determine if inherent characteristics of these bacteria have the capacity to act in a productive immune modulating fashion. Previous analyses of immunomodulating activities of 88 Mycobacterium strains provided useful insights regarding the utility of various strains as potential candidates for immunotherapy of cancer in reference to their pathogenicity and growth rate (Yuksel et al., 2011). However, the use of the monocytic cell line for immune challenges and a limited number of immune parameters measured in this study, namely IL-12 and TNF-α, limited its applications. In this Example, the immunostimulating properties of seven Mycobacterium strains was evaluated using PBMC cultures from healthy donors and compared them to the responses to B. subtilis strain.

The production of two cytokines, IL-6 and IL-8 as well as chemokines MIP-la and MIP-1β was measured in immune challenges using the parameters previously measured in previous fibromyalgia studies (Behm, et al., 2012; Wallace et al., 2014). Two Mycobacterium strains, M. smegmatis and M. agri, were identified to be the most effective in inducing PBMC immune responses. It has been shown that M. smegmatis preparations induce a potent immune response (Sweeney et al., 2011), display high anti-tumor activity in a mouse model (Kuhn, et al., 2013) and were effective in cancer immunotherapy studies (Ribi et al., 1976). Consistent with the results of this study, live M. smegmatis cells were capable of inducing production of IL-6, IL-8 and other cytokines in neutrophil cultures (Faldt et al., 2002).

M. brumae is yet another promising candidate for immunotherapy. High anti-tumor and immunomodulatory activities of this Mycobacterium strain has been demonstrated (Noguera-Ortega et al., 2016b). However, the results is this study showed only moderate PBMC responses to this strain compared to M. smegmatis and M. agri (FIGS. 2A-2D). The production of cytokines IL-6 and IL-8 by immune cells in response to M. brumae were in agreement with previously reported studies (Linares et al., 2012; Noguera-Ortega et al., 2016a).

The absence of immune responses to M. phlei, M. tokaiense, M. aurum and M. obuense strains was somewhat surprising since these Mycobacterium strains showed significant TNF-alpha and IL-12 stimulation activities in a cultured cell line (Yuskel et al., 2011). M. obuense and M. phlei were active ingredients of vaccine preparations SRL 172 (O'Brien et al., 2004) and MCNA (Morales et al., 2015) which were investigated in a number of pre-clinical and clinical trials regarding the immunotherapy of cancer (Noguera-Ortega et al., 2020). These strains were also shown to induce immune responses in human cell challenges (Reader et al., 2001; Bazzi et al., 2015; Noguera-Ortega et al., 2016b; Bazzi et al., 2017). The lack of PBMC responses by these Mycobacterium preparations in the experiments in this Example was not due to either cell toxicity or immunosuppression which may have been brought about by high concentrations of heat-killed cells or possible reagent contaminations. Differences in bacterial preparations or using different types of human cells may account for the apparent discrepancy of the present results with previously reported studies.

It was also demonstrated that cellular immune responses are similar for different Mycobacterium strains suggesting that they engage the same activation mechanisms. In contrast, bacterial strains of different genera evoked distinct responses in immune cell cultures, manifested by different patterns of extracellular cytokine productions, consistent with previously reported studies (Jonsson, et al., 2012a; Jonsson, et al., 2012b). Comparisons of cytokine response patterns for various strains provided a useful tool in studying the immune effects of various pathogenic bacteria and determining bacterial strain identities.

The release of specific chemokines and cytokines can be especially valuable as it concerns diseases where immune deficiency exists, such as fibromyalgia, interstitial cystitis and chronic pain. If an immune-modulating intervention pathway were to be identified, various modalities of therapy with non-pathologic organisms could be achieved, thereby limiting any potential risk for adverse side effects. Sites of action can include various microbiomes including, but not limited, to the microbiome of the gastrointestinal tract and of the vagina. Mycobacteria preparations are generally safe and well tolerated (Bach-Griera, et. al., 2020; Nouioui and Dye, 2021). Resultantly, the benefits of such interventions can act in a positive fashion without generating significant risks. The methodology outlined in this example may provide a useful tool for studying immune effects of various bacterial species.

### REFERENCES

Bach-Griera M, Campo-Peres V, Barbosa S, Traserra S, Guallar-Garrido S, Moya-Anderico L, et al. Mycolicibacterium brumae is a safe and non-toxic immunomodulatory agent for cancer treatment. Vaccines 2020; 8(2): 198.
Bazzi, S., Modjtahedi, H., Mudan, S., Akle, C and Bahr, G. M. (2015) lmmunobiology 220: 1293-1304.
Bazzi, S., Modjtahedi, H., Mudan, S., Achkar, M., Akle, C and Bahr, G. M. (2017) Innate lmmun. (2017) 23(7):592-605.
Behm, F. G., Gavin, I. M., Karpenko, 0., Lindgren, V., Gaitonde, S., Gashkoff, P. A. and Gillis, B. S. (2012) Unique immunologic patterns in fbromyalgia. BMC Clin Pathol 12:25.
Brausi, M. (2008) Eur. Urol. Suppl. 7:615-617
Faldt, J., Dahlgren, C. and Ridell, M. (2002) APMIS 110:593-600.
Filion, M. C., Lepicier, P., Morales, A. and Philips, N.C. (1999) Br. J. Cancer 79(2):229-235.
Filion, M. C., Filion, B., Reader, S., Menard, S. and Philips, N.C. (2000) Cancer Immunol. lmmunohter. 49:325-334.
Fowler, D. W., Copier, J., Wilson, N., Dalgleish, A. G. and Bodman-Smith, M. D. (2012) Cancer Immunol. lmmunother. 61:535-547.
Freedman, K. E., Hill, J. L., Wei, Y., Vazquez, A. R., Grubb, D. S., Trotter, R. E., Wrigley, S. D., Johnson, S. A., Foster, M. T. amd Weir, T. L. (2021) Examining the Gastrointestinal and lmmunomodulatory Effects of the Novel Probiotic Bacillus subtilis DE111. Int. J. Mol. Sci. 22(5):2453
Jonsson, B., Ridel, M. and Wold, A. E. (2012a) Microbes Infect. 14:768-777.
Jonsson, B., Ridel, M. and Wold, A. E. (2012b) Microbes Infect. 14:1186-1195.
Kuhn, S., Hyde, E. J., Yang, J., Rich, F. J., Harper, J. L., Kirman, J. R. and Ronchese, F. (2013) J. immunol. 191:1984-1992.
Linares, C., Bernabeu, A., Luquin, M. and Valero-Guillen, P. L. (2012) Cord factors from atypical mycobacteria (Mycobacterium alvei, Mycobacterium brumae) stimulate the secretion of some pro-inflammatory cytokines of relevance in tuberculosis. Microbiology (Reading) 158(Pt.11):2878-2885.
Morales, A., Herr, H., steinberg, G., Given, R., Cohen, Z., Amrhein, J and Kamat, A. M. (2015) J Urol. 193: 1135-1143.
Noguera-Ortega, E., Rabanal, R. M., Secanella-Fandos, S., Torrents, E., Luquin, M., Julian, E. (2016a) J. Urol. 195:198-205.
Noguera-Ortega, E., Secanella-Fandos, S., Eraria, H., Gasión, J., Rabanal, R. M., Luquin, M., Torrents, E., Julian, E. (2016b) Nonpathogenic Mycobacterium brumae Inhibits Bladder Cancer Growth In Vitro, Ex Vivo, and In Vivo. Eur. Urol. Focus 2(1):67-76.
Noguera-Ortega, E., Guallar-Garrido, S. and Julian, E. (2020) Cancers 12(7):1802.
Nouioui I, Dye T. Heat-killed Mycolicibacterium aurum Aogashima: An environmental nonpathogenic Actinobacteria under development as a safe novel food ingredient. Food Sci Nutr. 2021; 9(9):4839-54.
Pettenati C, Ingersoll M A. Mechanisms of BCG immunotherapy and its outlook for bladder cancer. Nat Rev Urol. 2018; 15(10):615-625.
O'Brien, M. E. R., Anderson, H., Kaukel, E., O'Byrne, K., Pawlicki, M., von Pawel, J. and Reck, M. (2004) Ann. Oncol. 15:9006-914.
Ribi, E., Milner, K. C., Granger, D. L., Kelly, M. T., Yamamoto, K., Brehmer, W., Parker, R., Smith, R. F., and Strain S. M. Immunotherapy with nonviable microbial components (1976) Ann. N.Y. Acad. Sci. 277:228-38.
Reader, S., Menard, S., Filion, B., Filion, M. C. and Phillips, N.C. (2001) The Prostate 49:155-165.
Shah, S. M., Ravi Kumar, G. V., Brah, G. S., Santra, L. and Pawar, H. (2012) Differential Expression of Th1- and Th2-Type Cytokines in Peripheral Blood Mononuclear Cells of Murrah Buffalo (Bubalus Bubalis) on TLR2 Induction by B. Subtilis Peptidoglycan. Asian-Australas. J. Anim. Sci. 25(7):1021-1028.
Sweeney, K. A., Dao, D. N., Goldberg, M. F., Hsu, T., Venkataswamy, M. M., Henao-Tamayo, M., Ordway, D., Sellers, R. S., Jain, P., Chen, B., Chen, M., Kim, J., Lukose, R., Chan, J., Orme, I. M., Porcelli, S. A. and Jacobs, W. R. (2011) Nat. Med. 17(10):1261-1268.
Witjes, J. A., Palou, J., Soloway, M., Lamm, D., Brausi, M., Spermon, J. R., Persad, R., Buckley, R., Akaza, H., Colombe!, M. and Bohle, A. (2008) Eur. Ural. Suppl. 7:667-674
Wallace, D. J., Gavin, I. M., Karpenko, 0., Barkhordar, F. and Gillis, B. S. (2014) Cytokine and chemokine profles in fbromyalgia, rheumatoid arthritis and systemic lupus erythematosus: a potentially useful tool in differential diagnosis. Rheumatol. Int. 35(6):991-996
Yuksel, Z. S., Buber, E., Kocagoz, T., Alp, A., Saribas, Z. and Acan, N. L. (2011) J. Mol. Microbial. Biotechnol. 20:24-28.
Zheng, D., Liwinski, T. and Elinav, E. (2020) Interaction between microbiota and immunity in health and disease. Cell Res. 30:492-506.

All publications, patents and patent applications herein are incorporated by reference to the same extent as if each individual publication or patent application was specifically and individually indicated to be incorporated by reference. It is not an admission that any of the information provided herein is prior art or relevant to the presently claimed inventions, or that any publication specifically or implicitly referenced is prior art.

While the invention has been described in connection with specific embodiments thereof, the foregoing description has been given for clearness of understanding only and no unnecessary limitations should be understood therefrom. It will be understood that the description is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. Those skilled in the art will recognize, or be able to ascertain using no more than routine experimentation, many equivalents to the specific embodiments of the invention described herein. Such equivalents are intended to be encompassed by the following claims.

### FURTHER NUMBERED EMBODIMENTS OF THE DISCLOSURE

Other subject matter contemplated by the present disclosure is set out in the following numbered embodiments:

1. A method of modulating immune system function in a subject, comprising administering a composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof.

2. The method of embodiment 1, wherein the Mycobacterium is live-attenuated.

3. The method of embodiment 1, wherein the Mycobacterium is heat-killed.

4. The method of any one of the above embodiments, wherein the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023.

5. The method of any one of the above embodiments, wherein the composition is formulated as a nutritional supplement and/or post-biotic.

6. The method of embodiment 5, wherein the nutritional supplement and/or post-biotic is incorporated into a food or beverage.

7. The method of embodiment 6, wherein the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks.

8. The method of any one of the above embodiments, wherein the composition comprises from 2×10⁷ to 5×10⁹ colony forming units (CFUs) of the Mycobacterium.

9. The method of any one of the above embodiments, wherein the composition comprises at least about 5×10⁶ CFU of the Mycobacterium.

10. The method of any one of embodiments 1-7, wherein the composition comprises 1 mg, 10 mg, 100 mg or 250 mg of the Mycobacterium.

11. The method of any one of the above embodiments, wherein the composition is administered in repeat doses.

12. The method of any one of the above embodiments, wherein the modulation in immune system function is evidenced by production and/or secretion of TH1 cytokines, upregulation of granzyme B or both as compared to a control.

13. The method of any one of the above embodiments, wherein the modulation in immune system function is evidenced by production and/or secretion of cytokines selected from the group consisting of interluekin-6 (IL-6), interluekin-8 (IL8), macrophage inflammatory protein-1 alpha (MIP-1alpha), macrophage inflammatory protein-1 beta (MIP-1beta) and any combination thereof as compared to a control.

14. The method of embodiment 12 or 13, wherein the control is a subject that has not been administered the composition.

15. The method of any one of the above embodiments, wherein the subject suffers from a disease or disorder caused by a deficiency or abnormality in immune system function.

16. The method of any one of the above embodiments, wherein the subject suffers from a disease or disorder selected from the group consisting of fibromyalgia, chronic fatigue syndrome, chronic pain, cancer interstitial cystitis, a primary immune deficiency disease (PIDD), depression, anxiety, diabetes, autoimmune diseases and inflammatory diseases.

17. A composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof.

18. The composition of embodiment 17, wherein the Mycobacterium is live-attenuated.

19. The composition of embodiment 17, wherein the Mycobacterium is heat-killed.

20. The composition of any one of embodiments 17-19, wherein the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023.

21. The composition of any one of embodiments 17-20, wherein the composition is formulated as a nutritional supplement and/or post-biotic.

22. The composition of embodiment 21, wherein the nutritional supplement and/or post-biotic is incorporated into a food or beverage.

23. The composition of embodiment 22, wherein the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks.

24. The composition of any one of embodiments 17-23, wherein the composition comprises from 2×10⁷ to 5×10⁹ colony forming units (CFUs) of the Mycobacterium.

25. The composition of any one of embodiments 17-24, wherein the composition comprises at least about 5×10⁶ CFU of the Mycobacterium.

26. The compositions of any one of embodiments 17-23, wherein the composition comprises 1 mg, 10 mg, 100 mg or 250 mg of the Mycobacterium.

What is claimed is:

1. A method of modulating immune system function in a subject, comprising administering a composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof.

2. The method of claim 1, wherein the Mycobacterium is live-attenuated.

3. The method of claim 1, wherein the Mycobacterium is heat-killed.

4. The method of any one of the above claims, wherein the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023.

5. The method of any one of the above claims, wherein the composition is formulated as a nutritional supplement and/or post-biotic.

6. The method of claim 5, wherein the nutritional supplement and/or post-biotic is incorporated into a food or beverage.

7. The method of claim 6, wherein the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks.

8. The method of any one of the above claims, wherein the composition comprises from 2×10⁷ to 5×10⁹ colony forming units (CFUs) of the Mycobacterium.

9. The method of any one of the above claims, wherein the composition comprises at least about 5×JAP CFU of the Mycobacterium.

10. The method of any one of claims 1-7, wherein the composition comprises 1 mg, 10 mg, 100 mg or 250 mg of the Mycobacterium.

11. The method of any one of the above claims, wherein the composition is administered in repeat doses.

12. The method of any one of the above claims, wherein the modulation in immune system function is evidenced by production and/or secretion of TH1 cytokines, upregulation of granzyme B or both as compared to a control.

13. The method of any one of the above claims, wherein the modulation in immune system function is evidenced by production and/or secretion of cytokines selected from the group consisting of interluekin-6 (IL-6), interluekin-8 (IL8), macrophage inflammatory protein-1 alpha (MIP-1alpha), macrophage inflammatory protein-1 beta (MIP-1beta) and any combination thereof as compared to a control.

14. The method of claim 12 or 13, wherein the control is a subject that has not been administered the composition.

15. The method of any one of the above claims, wherein the subject suffers from a disease or disorder caused by a deficiency or abnormality in immune system function.

16. The method of any one of the above claims, wherein the subject suffers from a disease or disorder selected from the group consisting of fibromyalgia, chronic fatigue syndrome, chronic pain, cancer interstitial cystitis, a primary immune deficiency disease (PIDD), depression, anxiety, diabetes, long COVID, brain fog, sleep disturbances or disorders, autoimmune diseases and inflammatory diseases.

17. A composition comprising a non-pathogenic strain of Mycobacterium selected from the group consisting of Mycobacterium agri, Mycobacterium phlei, Mycobacterium tokaiense, Mycobacterium smegmatis, Mycobacterium brumae, Mycobacterium aurum, Mycobacterium obuense and combinations thereof.

18. The composition of claim 17, wherein the Mycobacterium is live-attenuated.

19. The composition of claim 17, wherein the Mycobacterium is heat-killed.

20. The composition of any one of claims 17-19, wherein the non-pathogenic strain of Mycobacterium is a sub-strain selected from the group consisting of M. agri ATCC27406, M. phlei ATCC11758, M. tokaiense ATCC27282, M. smegmatis ATCC19420, M. brumae ATCC51384, M. aurum ATCC23366 and M. obuense, ATCC27023.

21. The composition of any one of claims 17-20, wherein the composition is formulated as a nutritional supplement and/or post-biotic.

22. The composition of claim 21, wherein the nutritional supplement and/or post-biotic is incorporated into a food or beverage.

23. The composition of claim 22, wherein the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks.

24. The composition of any one of claims 17-23, wherein the composition comprises from 2×10⁷ to 5×10⁹ colony forming units (CFUs) of the Mycobacterium.

25. The composition of any one of claims 17-24, wherein the composition comprises at least about 5×10⁶ (CFU of the Mycobacterium.

26. The compositions of any one of claims 17-23, wherein the composition comprises 1 mg, 10 mg, 100 mg or 250 mg of the Mycobacterium.
